(19) United States
(12) Patent Application Publication
(10) Pub. No.: US 2026/0355490 A1
(43) Pub. Date: Nov. 12, 2026

(54) ASEPTIC CONNECTOR SYSTEM

(71) Applicant: WATSON-MARLOW LIMITED, Falmouth Cornwall (GB)

(72) Inventors: Izzy BILLAU, Falmouth Cornwall (GB); Simon WOODS, Falmouth Cornwall (GB); Nick WHITE, Falmouth Cornwall (GB)

(73) Assignee: WATSON-MARLOW LIMITED, Falmouth Cornwall (GB)

(21) Appl. No.: 19/488,705

(22) PCT Filed: May 31, 2024
(86) PCT No.: PCT/EP2024/065056

§ 371 Date: Nov. 28, 2025

(30) Foreign Application Priority Data
May 31, 2023 (GB) 2308162.3

(51) Int. Cl. A61M 39/10; A61M 39/18; F16L 33/18

(52) U.S. Cl. CPC A61M 39/1011; A61M 39/18; F16L 33/18; A61M 2039/1072; F16L 2201/44

(57) ABSTRACT

An aseptic connector system includes a first connector and a second connector. The first connector includes a rotatable member rotatably mounted within a housing having a receiving chamber and an interconnecting passageway extending between a pair of openings. The rotatable member is rotatable between a closed position and an open position where the openings of the interconnecting passageway are aligned with a first fluid passageway and a connector port. The second connector includes a translatable member having a second fluid passageway and is translatable between a retracted position and a depressed position. The rotatable member can be rotated from the closed position to the open position, moving a first and/or a second sealing bung away the connector port and sealing the openings of the interconnecting passageway against the first and second fluid passageways, forming a continuous fluid pathway across the first and second connectors.

### CROSS-REFERENCE TO RELATED APPLICATIONS

This application is the U.S. national stage of PCT/EP2024/065056, filed May 31, 2024, which claims priority of Great Britain Patent Application No. GB 2308162.3, filed May 31, 2023, the contents of which are incorporated herein.

### TECHNICAL FIELD

The disclosure relates to an aseptic connector system.

### BACKGROUND

Sterile connectors for aseptic processing (commonly referred to as “aseptic connectors”) enable two lines of tubing to be joined while maintaining a sterile fluid pathway. This may be particularly important in medical, pharmaceutical and bioprocessing applications.

An end of each line of tubing may be provided with an aseptic connector which is configured to engage and mechanically connect with the opposing connector. Each aseptic connector is provided with a sealing arrangement which retains the sterility of the tubing (as well as any portions of the connector which form part of the fluid pathway) before, during and after their interconnection. For example, the end of each connector may be provided with a removable membrane which can be removed once the two connectors are interconnected, and the fluid pathway is sealed.

It is desirable to provide aseptic connectors which are simple and easy to operate in order to avoid user errors which could inadvertently compromise the sterility of the fluid pathway.

### SUMMARY

In accordance with an aspect of the disclosure, there is provided an aseptic connector system including a first connector and a second connector. The first connector includes: a housing which defines a cavity, the housing having a first fluid passageway and a connector port which open into the cavity; and a rotatable member rotatably mounted within the cavity. The rotatable member includes a receiving chamber formed in the rotatable member and an interconnecting passageway extending through the rotatable member between a pair of openings, wherein the receiving chamber is angularly offset from the openings of the interconnecting passageway; wherein the rotatable member is rotatable between a closed position in which the openings of the interconnecting passageway are covered and sealed by the housing and the receiving chamber is aligned with the connector port, and an open position in which the openings of the interconnecting passageway are aligned with the first fluid passageway and the connector port and the receiving chamber is angularly offset from the connector port. The second connector includes: a translatable member having a second fluid passageway which extends through the translatable member; wherein the translatable member is translatable between a retracted position and a depressed position. The first connector includes a first sealing bung disposed in the connector port and/or the second connector includes a second sealing bung coupled to the translatable member; wherein the first connector is configured to be mechanically connected to the second connector; wherein, with the rotatable member in the closed position, the translatable member is translatable from the retracted position to the depressed position in order to force the first and/or second sealing bungs through the connector port and into the receiving chamber formed in the rotatable member; and wherein the rotatable member can then be rotated from the closed position to the open position in order to move the first and/or second sealing bungs away from the connector port and such that the openings of the interconnecting passageway are sealed against the first and second fluid passageways so as to form a continuous fluid pathway across the first and second aseptic connectors.

In some embodiments, the first sealing bung extends from the connector port into the receiving chamber.

In some embodiments, opposing surfaces of two or more of the first sealing bung, the second sealing bung and the receiving chamber comprise complementary formations which engage when the translatable member is translated to the depressed position.

In some embodiments, an end surface of the translatable member is curved and forms a continuation of an inner surface of the housing when the translatable member is in the depressed position.

In some embodiments, an end surface of one of the first and second sealing bungs is curved and forms a continuation of an outer surface of the rotatable member when received in the receiving chamber.

In some embodiments, the first fluid passageway and the connector port are on opposite sides of the housing.

In some embodiments, the first and second fluid passageways and/or one or both of the openings of the interconnecting passageway are each provided with a seal.

In some embodiments, the or each seal is compressed in an axial direction of the seal when the rotatable member is in the closed position and expand in the axial direction when the rotatable member is in the open position to seal the interconnecting passageway against the first and second fluid passageways.

In some embodiments, the rotatable member comprises an actuation portion which extends externally to the housing so as to allow the rotatable member to be rotated.

In some embodiments, the first fluid passageway is sealed by an outer surface of the rotatable member in the closed position.

In some embodiments, the cavity is cylindrical.

In some embodiments, the second connector further comprises a connecting collar; wherein the first connector is configured to be mechanically connected to the second connector by engaging the connecting collar with the connector port; and wherein the translatable member is coupled to the connecting collar and translatable relative to the connecting collar.

In some embodiments, the second sealing bung is disposed in the connecting collar.

In some embodiments, the axis of rotation of the rotatable member passes through the interconnecting passageway.

In some embodiments, the rotatable member is rotated by 90 degrees to move between the closed position and the open position.

In some embodiments, the receiving chamber is angularly offset by 90 degrees from the openings of the interconnecting passageway.

In some embodiments, the interconnecting passageway, and first and second fluid passageways are offset from the axis of rotation.

In some embodiments, the rotatable member is rotated by 180 degrees to move between the closed position and the open position.

In some embodiments, the receiving chamber is angularly offset by 180 degrees from one of the openings of the interconnecting passageway.

### BRIEF DESCRIPTION OF DRAWINGS

For a better understanding of the disclosure, and to show more clearly how it may be carried into effect, reference will now be made, by way of example, to the accompanying drawings, in which:

FIG. 1 is a perspective view of an aseptic connector system according to an embodiment of the disclosure;

FIG. 2 is a perspective view of the aseptic connector system with an actuation portion removed;

FIG. 3 is a cross-sectional view of the aseptic connector system;

FIG. 4 is a perspective view of the aseptic connector system in a mechanically connected, but closed configuration;

FIG. 5 is a cross-sectional view of the aseptic connector system in a mechanically connected, but closed configuration;

FIG. 6 is a perspective view of the aseptic connector system in first stage of a transition between the closed configuration and an open configuration;

FIG. 7 is a cross-sectional view of the aseptic connector system in first stage of a transition between the closed configuration and an open configuration;

FIG. 8 is a perspective view of the aseptic connector system in second stage of a transition between the closed configuration and the open configuration;

FIG. 9 is a cross-sectional view of the aseptic connector system in second stage of a transition between the closed configuration and the open configuration;

FIG. 10 is a perspective view of the aseptic connector system in the open configuration;

FIG. 11 is a cross-sectional view of the aseptic connector system in the open configuration;

FIG. 12 is a perspective view of an aseptic connector system according to another embodiment of the disclosure;

FIG. 13 is a top and view of the aseptic connector system;

FIG. 14 is a cross-sectional view of the aseptic connector system;

FIG. 15 is a top view of the aseptic connector system in a mechanically connected, but closed configuration;

FIG. 16 is a cross-sectional views of the aseptic connector system in a mechanically connected, but closed configuration;

FIG. 17 is a top view of the aseptic connector system in an open configuration; and

FIG. 18 is a cross-sectional view of the aseptic connector system in an open configuration

### DETAILED DESCRIPTION

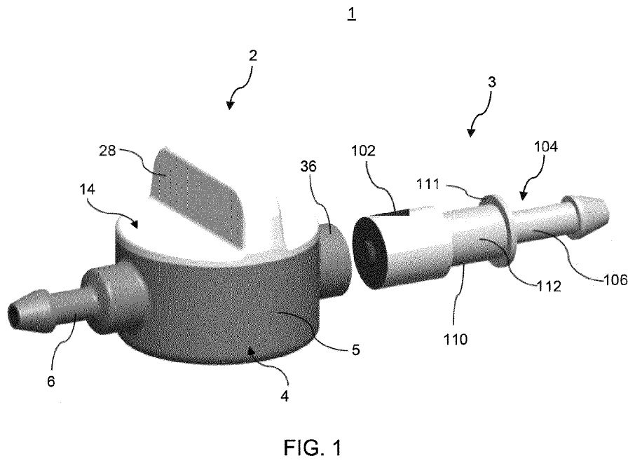

FIGS. 1 to 3 show an aseptic connector system 1 according to an embodiment of the disclosure. The aseptic connector system 1 comprises a first connector 2 and a second connector 3.

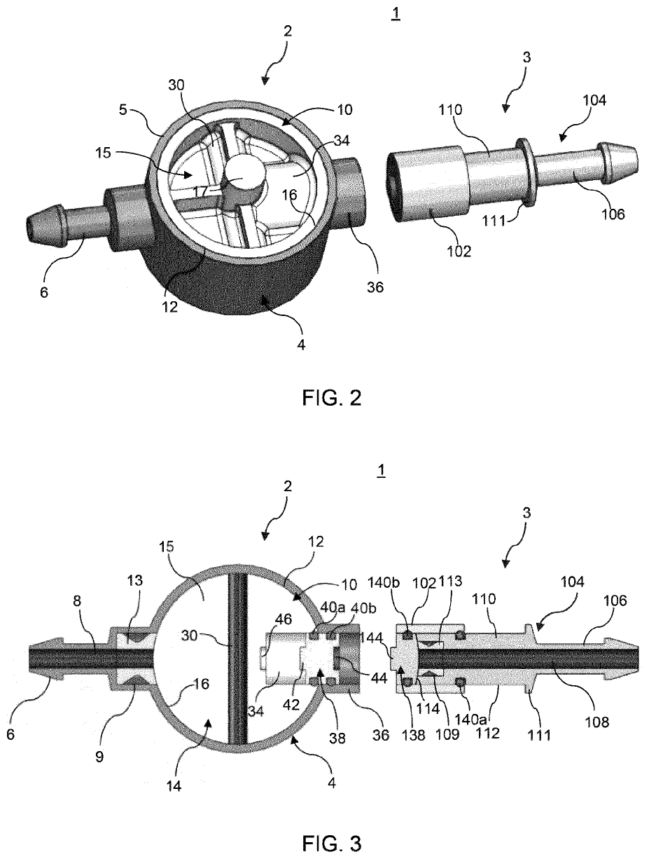

The first connector 2 comprises a housing 4. The housing 4 comprises a barb fitting 6 for insertion into a tube. As shown in FIG. 3, a fluid passageway 8 extends through the fitting 6. The housing 4 comprises a cylindrical wall 5 and a pair of end walls which define an inner cavity 10, as seen in FIGS. 2 and 3. The fluid passageway 8 passes through the cylindrical wall 5 to form an opening in an inner circumferential surface 12 of the cylindrical wall 5.

The fluid passageway 8 comprises a counterbore section 9 where it opens into the circumferential surface 12. The counterbore section 9 receives a seal 13. The seal 13 has an hourglass-like profile which comprises a section that tapers (i.e., reduces in diameter) from either end towards the middle in an axial direction. This structure allows the seal 13 to be easily compressed in an axial direction and to return quickly to its relaxed state. The counterbore section 9 has a depth which is less than the length of the seal 13 when in the relaxed state such that the seal 13 partially projects from the counterbore section 9 when in the relaxed state.

The seal 13 has a maximum outer diameter which corresponds to an inner diameter of the counterbore section 9 and an inner diameter which corresponds to the inner diameter of the fluid passageway 8. Accordingly, the seal 13 forms a continuation of the fluid passageway 8 with no significant deviation in the diameter.

The first connector 2 further comprises a rotatable member 14 which comprises a body portion 15 and an actuation knob 28 (or any other actuation portion) which is connected to the body portion 15 by a shaft 17. The body portion 15 is received within the cavity 10 of the housing 4. The body portion 15 is cylindrical and comprises a circumferential surface 16 which conforms to the inner circumferential surface 12 of the cylindrical wall 5. The rotatable member 14 is rotatable relative to the housing 4 about an axis of rotation which extends through the axis of the shaft 17 and through a centre of the housing 4.

The shaft 17 extends from the centre of a radial end surface of the body portion 15 and passes through a complementary hole provided in one of the end walls of the housing 4. The actuation knob 28 is thus disposed externally to the housing 4. The actuation knob 28 can be grasped and used to rotate the rotatable member 14 within the inner cavity 10 between a first, closed position and a second, open position, as will be described in further detail below.

The body portion 15 further comprises an interconnecting passageway 30 which extends through the axis of the body portion 15 (i.e., the axis of rotation) and has an opening at either end at diametrically opposed positions in the circumferential surface 16.

The body portion 15 further comprises a receiving chamber 34. The receiving chamber 34 extends into the body portion 15 from the circumferential surface 16. The receiving chamber 34 is angularly offset from the interconnecting passageway 30. In this example, the receiving chamber 34 is perpendicular to the interconnecting passageway 30. In other words, the receiving chamber 34 is offset from the openings of the interconnecting passageway 30 by 90 degrees around the circumferential surface 16 and thus is located midway between the openings around the circumferential surface 16. The receiving chamber 34 extends only partially into the body portion 15 and so does not intersect the interconnecting passageway 30.

The housing 4 further comprises a connector port 36 for receiving the second connector 3, as will be described further below. The connector port 36 passes through the cylindrical wall 5 to form an opening in the inner circumferential surface 12 of the cylindrical wall 5. The connector port 36 and the fitting 6 are located at diametrically opposed positions. The connector port 36 corresponds to the receiving chamber 34.

As shown in FIG. 3, in the first, closed position, the receiving chamber 34 is aligned with the connector port 36. A first sealing bung 38 is located in the connector port 36. The first sealing bung 38 blocks the connector port 38 and thus prevents contaminants from entering the cavity 10. As shown, the first sealing bung 38 extends partially into the receiving chamber 34. Accordingly, the first sealing bung 38 also prevents rotation of the rotatable member 14 relative to the housing 4 in this position.

The first sealing bung 38 is generally cylindrical and comprises are cylindrical outer surface and first and second end surfaces. A pair of O-rings 40a, 40b are provided in grooves formed in the cylindrical outer surface. The O-rings 40a, 40b (and grooves) are spaced from one another in an axial direction of the first sealing bung. The first end surface comprises a circular projection 42 and the second end surface comprises a circular recess 44. The first sealing bung 38 is received in the connector port 36 with the first end surface facing inwards (towards the cavity 10) and the second end surface facing outwards (away from the cavity 10). An end surface of the receiving chamber 34 also comprises a circular recess 46 which is complementary to the circular projection 42 of the first sealing bung 38.

The second connector 3 comprises a connecting collar 102 and a translatable member 104.

The translatable member 104 comprises a barb fitting 106 for insertion into a tube and a plunger portion 110 which extends from the barb fitting 106. As shown in FIG. 3, a fluid passageway 108 extends through the fitting 106 and the plunger portion 110 along an axial length of the translatable member 104.

The connecting collar 102 is cylindrical and is open at either end. The plunger portion 110 is received within the connecting collar 102 and is translatable relative to the connecting collar 102 in an axial direction.

The plunger portion 110 comprises a cylindrical wall 112 and an end wall 114. A rim 111 projects from the plunger portion 110 at an end adjacent to the fitting 106. A groove extends around the cylindrical wall 112 which receives an O-ring 140a that engages and seals against an inner surface of the connecting collar 102. The fluid passageway 108 forms an opening in the end wall 114.

The fluid passageway 108 comprises a counterbore section 109 where it opens into the end wall 114. The counterbore section 109 receives a seal 113. The seal 113 corresponds to the seal 13 described previously. The counterbore section 109 has a depth which is less than the length of the seal 113 when in the relaxed state such that the seal 113 partially projects from the counterbore section 109 when in the relaxed state.

A second sealing bung 138 is located in the connecting collar 102. The second sealing bung 138 blocks the connecting collar 102 and thus prevents contaminants from entering.

The second sealing bung 138 is received in the connecting collar 102 with a first end surface facing inwards (towards the plunger portion 110) and a second end surface facing outwards (away from the plunger portion 110). The second end surface is provided with a circular projection 144 which is complementary to the circular recess 44 provided in the first sealing bung 38. The first end surface is curved. The curvature of the first end surface corresponds to the curvature of the circumferential surface 16 of the rotatable member 14. The first end surface abuts against the end wall 114 of the plunger portion 110. The end wall 114 of the plunger portion 110 is also curved and has a complementary curvature which corresponds to the curvature of the inner circumferential surface 12 of the housing 4. The first end surface of the second sealing bung 138 is therefore able to seal against the end wall 114 of the plunger portion 110 and block the fluid passageway 108.

A groove extends around an outer surface of the second sealing bung 138 which receives an O-ring 140b that engages and seals against an inner surface of the connecting collar 102.

The first and second connectors 2, 3 may be mechanically connected to one another by a suitable interconnection mechanism formed between the connector port 36 and the connecting collar 102. For example, the interconnection mechanism may comprise a pair of cantilevered beams each having an opening (female element) which are provided at diametrically opposed sides of the connector port 36. The connecting collar 102 may comprise a pair of complementary retaining hooks (i.e., barbs) (male element) which are received in the openings of the cantilevered beams to form a snap fit connection with the hooks preventing withdrawal. In other examples, the positions of the male and female elements may be reversed or each connector 2, 3 may contain a male and a female element which engage with corresponding female and male elements on the opposing connector 2, 3. Alternatively, the connecting collar 102 may be rotated into engagement with the connector port 36 via a suitable thread or other arrangement. The interconnection mechanism connects and seals the connecting collar 102 of the second connector 3 to the connector portion 36 of the first connector 2.

The first and second connectors 2, 3 are assembled in a first, closed configuration.

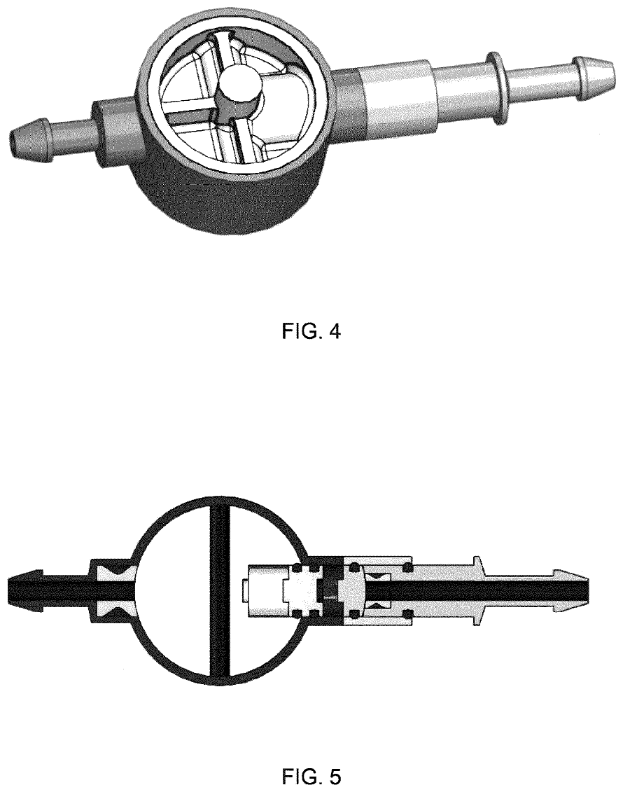

As shown in FIGS. 4 and 5, in this configuration, the interconnecting passageway 30 of the first connector 2 is perpendicular to the fluid passageways 8, 108 and thus the ends of the interconnecting passageway 30 are sealed by the circumferential surface 12 of the housing 4. The fluid passageway 8 of the first connector 2 is sealed by the circumferential surface 16 of the rotatable member 14 and the seal 13 is axially compressed back into the counterbore section 9.

In the closed configuration, the plunger portion 110 is in a retracted position such that second sealing bung 138 is held in the connecting collar 102 with its second end surface substantially flush with the end of the connecting collar 102 (as shown, in this example, the circular projection 144 projects out of the connecting collar 102). The fluid passageway 108 of the second connector 3 is sealed by the second sealing bung 138 and the seal 113 is axially compressed back into the counterbore section 109.

In this configuration, while the first and second connectors 2, 3 are mechanically connected, they remain fluidically disconnected with no fluid flow being possible between their respective fluid passageways 8, 108.

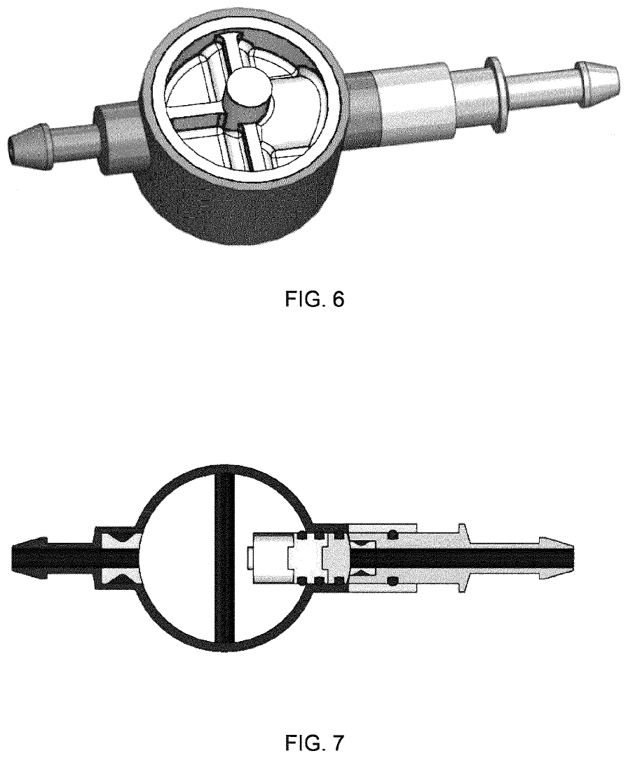

In order to connect their fluid passageways 8, 108, the plunger portion 110 is depressed such that the plunger portion 110 translates axially through the connecting collar 102 until the rim 111 abuts against the connecting collar 102. As shown in FIGS. 6 and 7, the initial translation of the plunger portion 110 causes the second sealing bung 138 to abut against the first sealing bung 38 with the circular projection 144 of the second sealing bung 138 being received in the circular recess 44 of the first sealing bung 38.

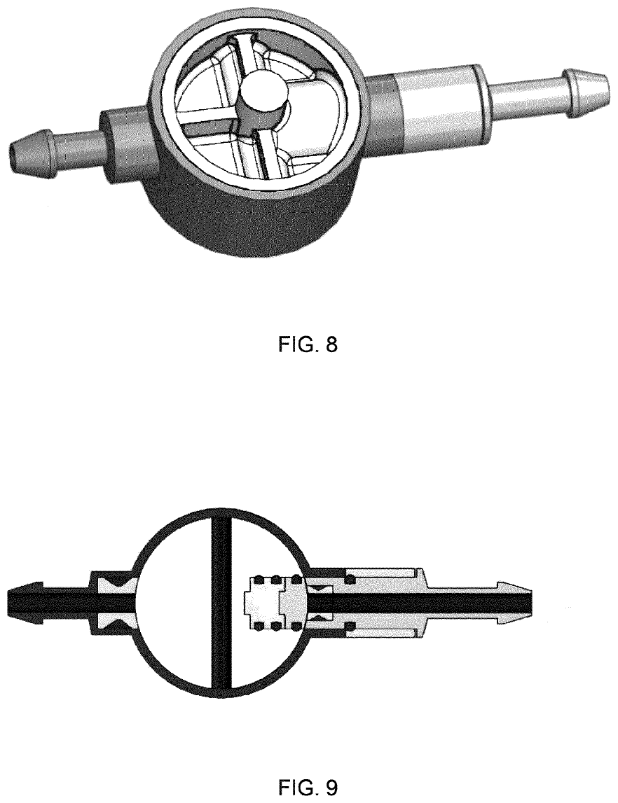

As shown in FIGS. 8 and 9, the further movement of the plunger portion 110 forces the first and second sealing bungs 38, 138 into the receiving chamber 34. The circular projection 42 of the first sealing bung 38 is received in the circular recess 46 at the base of the receiving chamber 34.

The end wall 114 of the plunger portion 110 forms a continuation of the inner circumferential surface 12 of the housing 4 and the first end surface of the second sealing bung 138 forms a continuation of the circumferential surface 16 of the rotatable member 14.

In this position, the fluid passageways 8, 108 continue to be blocked by the circumferential surface 16 of the rotatable member 14 and the second sealing bung 138 respectively. Similarly, the openings of the interconnecting passageway 30 continue to be sealed by the inner circumferential surface 12 of the housing 4.

With the first and second sealing bungs 38, 138 received in the receiving chamber 34, the rotatable member 14 can now be freely rotated using the actuator knob 28.

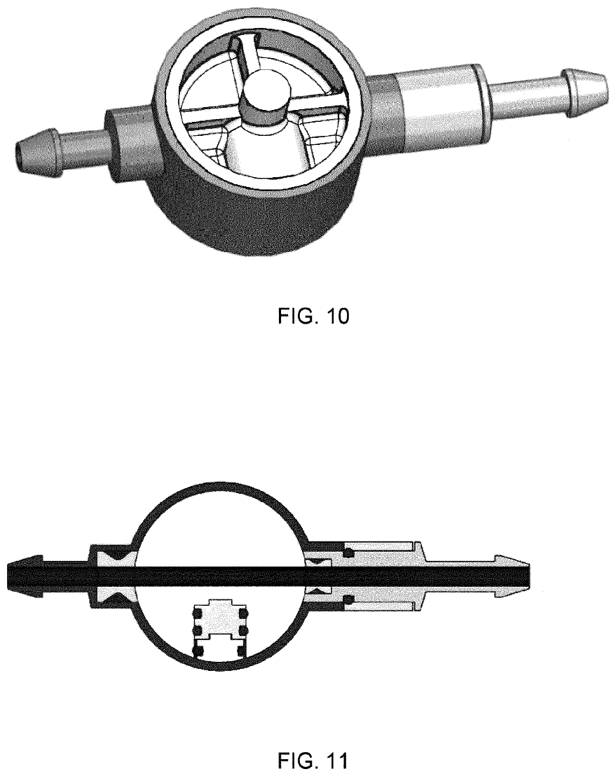

The actuator knob 28 can be rotated in a clockwise direction (although in other examples, it may be rotated in an anticlockwise direction) by 90 degrees, as shown in FIGS. 10 and 11. The rotation of the actuator knob 28 rotates the body portion 15 and the first and second sealing bungs 38, 138 are carried away from the plunger portion 110 in the receiving chamber 34.

With the actuator knob 28 rotated by 90 degrees, the interconnecting passageway 30 now extends between the fluid passageways 8, 108 via the seals 13, 113. Fluid can therefore pass across the fluid passageways 8, 108 of the first and connectors 2,3 via the interconnecting passageway 30 in this second, open configuration.

The plunger portion 110 may be held captive by the rotatable member 14 when in the open configuration. For example, the circumferential surface 16 may comprise a slot which receives the end of the plunger portion 110 and prevents withdrawal.

It will be appreciated that the surfaces which are exposed prior to connection of the first and second connectors 2, 3 are not in contact with the fluid passing through the first and second connectors 2, 3. Accordingly, any contaminants which may have come into contact with these surfaces cannot be introduced into the fluid flow.

In order to disconnect the first and second connectors 2, 3, the procedure can be reversed with the actuator knob 28 first being rotated in an anticlockwise direction (although in other examples, it may be rotated in a clockwise direction) and the plunger portion 110 then being retracted. The plunger portion 110 and the second sealing bung 138 may be provided with cooperating formations (such as grooves and ribs) which cause the second sealing bung 138 to engage with the plunger portion 110 when the actuator knob 28 is rotated back to the closed position such that the second sealing bung 138 moves with the plunger portion 110 as it is retracted. Likewise, the first sealing bung 38 may engage with the second sealing bung 138 such that it also translates with the second sealing bung 138 back to its original position. A formation in the receiving chamber 34 or connector port 36 may prevent further movement of the first sealing bung 38 such that it detaches from the second sealing bung 138 when in its original position and is not withdrawn further.

The first and second connectors 2, 3 are now in the closed configuration, as shown in FIGS. 4 and 5 and the first and second connectors 2, 3 can be mechanically disconnected from one another.

The housing 4 and rotatable member 14 and/or translatable member 104 and connecting collar 102 may have cooperating catch elements which retain the rotatable member 14 and/or translatable member 104 in the closed and/or open configurations and which may be released by user action. For example, the catch elements may retain the rotatable member 14 until a predetermined force is applied to the actuator knob 28 or may require a button or other element to be depressed or otherwise activated to release the rotatable member 14. Similarly, the catch elements may retain the translatable member 104 until a predetermined force is applied to the plunger portion 110 or may require a button or other element to be depressed or otherwise activated to release the translatable member 104.

The first and/or second connectors 2, 3 may be provided with one or more visual indicators which identify when the first and second connectors 2, 3 are in the open and/or closed configuration. For example, the circumferential surface 12 of the housing 4 may comprise a window through which the circumferential surface 16 of the rotatable member 14 can be seen. The circumferential surface 16 of the rotatable member 14 may be provided with a first visual indicator and a second visual indicator. Similarly, the connecting collar 102 may comprise a window through which the circumferential surface 112 of the plunger portion 110 can be seen. The circumferential surface 112 of the plunger portion 110 may be provided with a first visual indicator and a second visual indicator.

The first visual indicator is positioned such that it is visible through the window when the rotatable/translatable member is in the first, closed position and the second visual indicator is positioned such that it is visible through the window when the rotatable/translatable member is in the second, open position. Accordingly, the window and visual indicators provide feedback to a user regarding the current state of the connector 2, 3. In other examples, only a single visual indicator may be provided which confirms that the connector 2, 3 is in one of the open and closed configurations and the absence of the visual indicator is used to demonstrate that the connector 2, 3 is in the other of the open and closed configurations. Further, the or each visual indicator may be provided in a different location. For example, the or each visual indicator may be provided on the outer surface of the housing 4 in a position which is selectively obscured/exposed by the actuation knob 28 or on the circumferential surface 112 of the plunger portion 110 which is selectively obscured/exposed by the connecting collar 102.

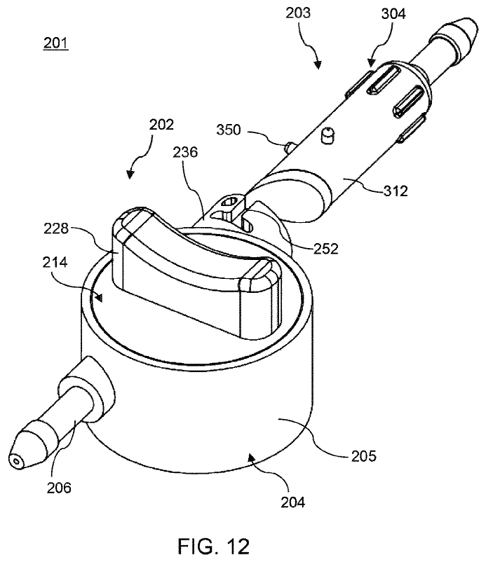

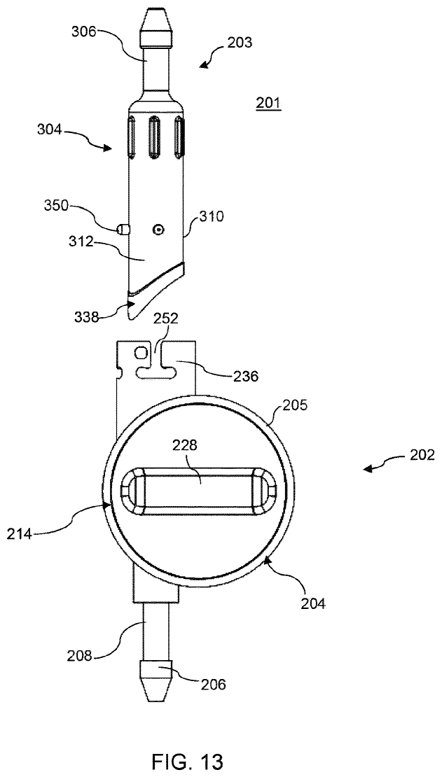

FIGS. 12 to 14 show an aseptic connector system 201 according to an embodiment of the disclosure. The aseptic connector system 201 is similar to the aseptic connector system 201 described previously and comprises a first connector 202 and a second connector 203.

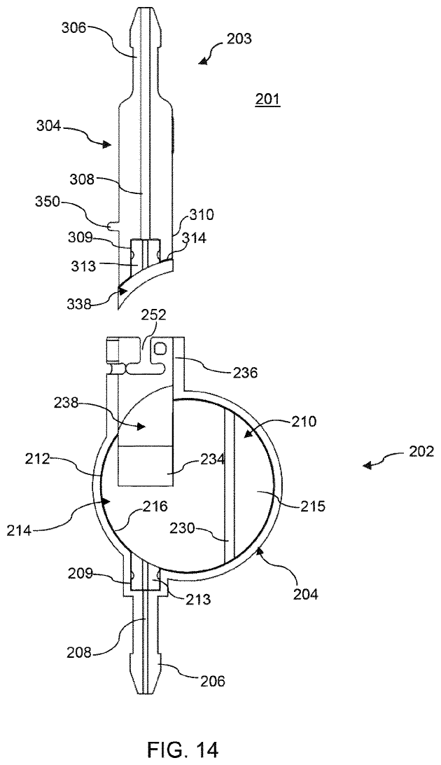

The first connector 202 comprises a housing 204. The housing 204 comprises a barb fitting 206 for insertion into a tube. As shown in FIG. 14, a fluid passageway 208 extends through the fitting 206. The housing 204 comprises a cylindrical wall 205 and a pair of end walls which define an inner cavity 210, as seen in FIG. 14. The fluid passageway 208 passes through the cylindrical wall 205 to form an opening in an inner circumferential surface 212 of the cylindrical wall 205.

The fluid passageway 208 comprises a counterbore section 209 where it opens into the circumferential surface 212. The counterbore section 209 receives a seal 213. The seal 213 corresponds to the seals 13, 113 described previously.

The first connector 202 further comprises a rotatable member 214 which comprises a body portion 215 and an actuation knob 228 (or any other actuation portion) which is connected to the body portion 215 by a shaft. The body portion 215 is received within the cavity 210 of the housing 204. The body portion 215 is cylindrical and comprises a circumferential surface 216 which conforms to the inner circumferential surface 212 of the cylindrical wall 205. The rotatable member 214 is rotatable relative to the housing 204 about an axis of rotation which extends through the axis of the shaft and through a centre of the housing 204.

The actuation knob 228 is disposed externally to the housing 204. The actuation knob 228 can be grasped and used to rotate the rotatable member 214 within the inner cavity 210 between a first, closed position and a second, open position, as will be described in further detail below.

The body portion 215 further comprises an interconnecting passageway 230. In this embodiment, the interconnecting passageway 230 does not extend through the axis of the body portion 215 (i.e., the axis of rotation) and instead is offset. The openings in the circumferential surface 216 at either end of the interconnecting passageway 230 therefore are not strictly diametrically opposed but instead define a chord which is not the diameter (i.e., it has a length that is less than the diameter). The interconnecting passageway 230 therefore divides the circle of the body portion 215 into a major segment and a minor segment.

The body portion 215 further comprises a receiving chamber 234. The receiving chamber 234 extends into the body portion 215 from the circumferential surface 216. The receiving chamber 234 is also offset from the axis of rotation, as well as from the interconnecting passageway 230. The receiving chamber 234 is offset from one of the openings of the interconnecting passageway 230 by 180 degrees around the circumferential surface 216. The receiving chamber 234 extends only partially into the body portion 215 and so does not intersect the interconnecting passageway 230.

The housing 204 further comprises a connector port 236 for receiving the second connector 203, as will be described further below. The connector port 236 passes through the cylindrical wall 205 to form an opening in the inner circumferential surface 212 of the cylindrical wall 205. The connector port 236 and the fitting 206 are located at opposite sides of the housing 204. The connector port 236 and the fitting 206 are offset from the axis of rotation such that a line extending between them defines a chord which is not the diameter. The connector port 236 corresponds to the receiving chamber 234.

As shown in FIG. 14, in the first, closed position, the receiving chamber 234 is aligned with the connector port 236. A first sealing bung 238 is located in the connector port 236. The first sealing bung 238 blocks the connector port 238 and thus prevents contaminants from entering the cavity 210. As shown, the first sealing bung 238 extends partially into the receiving chamber 234. Accordingly, the first sealing bung 238 also prevents rotation of the rotatable member 214 relative to the housing 204 in this position. The second connector 203 comprises a translatable member 304.

The translatable member 304 comprises a barb fitting 306 for insertion into a tube and a plunger portion 310 which extends from the barb fitting 306. As shown in FIG. 14, a fluid passageway 308 extends through the fitting 306 and the plunger portion 310 along an axial length of the translatable member 304.

The plunger portion 310 comprises a cylindrical wall 312 and an end wall 314. The fluid passageway 308 forms an opening in the end wall 314.

The fluid passageway 308 comprises a counterbore section 309 where it opens into the end wall 314. The counterbore section 309 receives a seal 313. The seal 313 corresponds to the seals 13, 113, 213 described previously.

A second sealing bung 338 is attached to the end wall 314 of the plunger portion 310. The second sealing bung 338 covers the end wall 314 and the seal 313. The second sealing bung 338 thus seals the fluid passageway 308 and prevents contaminants from entering.

The second sealing bung 338 is attached to the end wall 314 with a first end surface facing inwards (towards the plunger portion 310) and a second end surface facing outwards (away from the plunger portion 310). The first and second end surfaces are curved. The curvature of the first end surface corresponds to the curvature of the circumferential surface 216 of the rotatable member 214. The end wall 314 of the plunger portion 310 is also curved and has a complementary curvature which corresponds to the curvature of the inner circumferential surface 212 of the housing 204.

In this example, the second connector 203 does not comprise a separate connecting collar and the second connector 203 is connected directly to the first connector 202 by the translatable member 304.

In particular, the plunger portion 310 comprises a plurality of pins 350 which project from the cylindrical wall 312. The pins 350 are received in complementary slots 252 formed in the connector port 236. The slots 252 are T-shaped (in other examples, they may be L-shaped) and are arranged such that the pins 350 are received by translating the plunger portion 310 axially into the connector port 236. The second connector 203 can then be rotated (i.e., twisted) such that the pins 350 lock into position in the slots 252, thereby preventing withdrawal of the plunger portion 310 from the connector port 236.

The first and second connectors 202, 203 are assembled in a first, closed configuration.

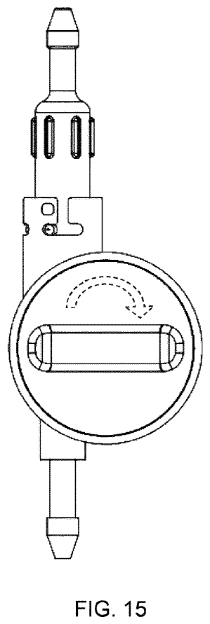

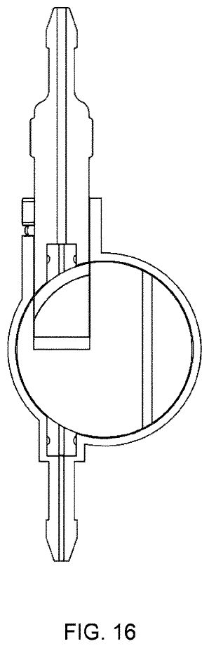

As shown in FIGS. 15 and 16, in this configuration, the interconnecting passageway 230 of the first connector 202 is offset from the fluid passageways 208, 308 and thus the ends of the interconnecting passageway 230 are sealed by the circumferential surface 212 of the housing 204. The fluid passageway 208 of the first connector 202 is sealed by the circumferential surface 216 of the rotatable member 214 and the seal 213 is axially compressed back into the counterbore section 209.

The fluid passageway 308 of the second connector 203 is sealed by the second sealing bung 338 and the seal 313 is axially compressed back into the counterbore section 309.

The plunger portion 310 is forced into the connector port 236. As shown in FIGS. 15 and 16, the translation of the plunger portion 310 causes the second sealing bung 338 to abut against the first sealing bung 238. The further movement of the plunger portion 310 forces the first and second sealing bungs 238, 338 into the receiving chamber 234.

The end wall 314 of the plunger portion 310 forms a continuation of the inner circumferential surface 212 of the housing 204 and the first end surface of the second sealing bung 338 forms a continuation of the circumferential surface 216 of the rotatable member 214.

In this configuration, while the first and second connectors 202, 203 are mechanically connected, they remain fluidically disconnected with no fluid flow being possible between their respective fluid passageways 208, 308.

With the first and second sealing bungs 238, 338 received in the receiving chamber 234, the rotatable member 214 can now be freely rotated using the actuator knob 228.

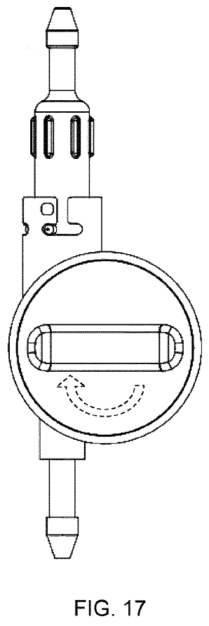

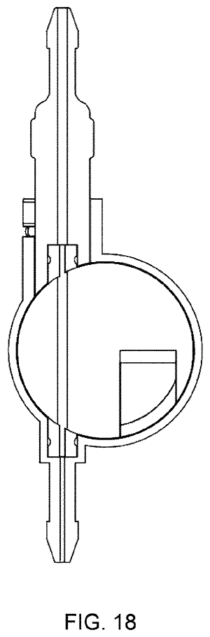

The actuator knob 228 can be rotated in a clockwise direction (although in other examples, it may be rotated in an anticlockwise direction) by 180 degrees, as shown in FIGS. 17 and 18. The rotation of the actuator knob 228 rotates the body portion 215 and the first and second sealing bungs 238, 338 are carried away from the plunger portion 310 in the receiving chamber 234.

With the actuator knob 228 rotated by 180 degrees, the interconnecting passageway 230 now extends between the fluid passageways 208, 308 via the seals 213, 313. Fluid can therefore pass across the fluid passageways 208, 308 of the first and connectors 202, 203 via the interconnecting passageway 230 in this second, open configuration.

The plunger portion 310 may be held captive by the rotatable member 214 when in the open configuration. For example, the circumferential surface 216 may comprise a slot which receives the end of the plunger portion 310 and prevents withdrawal.

It will be appreciated that the surfaces which are exposed prior to connection of the first and second connectors 202, 203 are not in contact with the fluid passing through the first and second connectors 202, 203. Accordingly, any contaminants which may have come into contact with these surfaces cannot be introduced into the fluid flow.

In order to disconnect the first and second connectors 202, 203, the procedure can be reversed with the actuator knob 228 first being rotated in an anticlockwise direction (although in other examples, it may be rotated in a clockwise direction) and the plunger portion 310 then being retracted. The plunger portion 310 and the second sealing bung 338 may be provided with cooperating formations (such as grooves and ribs) which cause the second sealing bung 338 to engage with the plunger portion 310 when the actuator knob 228 is rotated back to the closed position such that the second sealing bung 338 moves with the plunger portion 310 as it is retracted. Likewise, the first sealing bung 238 may engage with the second sealing bung 338 such that it also translates with the second sealing bung 338 back to its original position. A formation in the receiving chamber 234 or connector port 236 may prevent further movement of the first sealing bung 238 such that it detaches from the second sealing bung 338 when in its original position and is not withdrawn further.

In the examples described above, the cavity 10, 210 and rotatable member 14, 214 have circular symmetry around an axis of revolution which is aligned with the rotational axis of the rotatable member 14, 214. It will be appreciated that the cavity 10, 210 and rotatable member 14, 214 need not be cylindrical and could be formed by another solid of revolution which has circular symmetry. For example, the cavity 10, 210 and rotatable member 14, 214 could be spherical, conical, biconical, etc.

In other examples, the first sealing bung 38, 238 may be located entirely in the connector port 36, 236 in the first, closed position and only forced into the receiving chamber 34, 234 under the actuation of the plunger portion 110, 310. The rotatable member 14, 214 may be prevented from rotating by other means. The first sealing bung 38, 238 may also be positioned such that it abuts against the second sealing bung 138, 338 when the first and second connectors are initially connected, without first requiring the plunger portion 110, 310 to be translated.

In other examples, one or more of the seals may not be expandable.

It will be appreciated that other examples may use different forms of seal which have a structure that is configured in order to reduce the compressive stiffness in an axial direction (i.e., beyond the mechanical properties of the material itself). For example, the seal may have a helical groove which runs around the outer surface of the seal. Further, seals may be provided in the openings of the interconnecting passageway 30, 230 as an alternative or in addition to the seals 13, 113, 213, 313.

It will be appreciated that in other examples, the first and second connectors may have an alternative fitting instead of the barb fitting described above. For example, one or both of the first and second connectors may be provided with a flange fitting.

In other examples, only one of the first and second connectors may be provided with a sealing bung and the other connector may be sealed in another way.

In other examples, the first and second connectors may be provided with a protective cover which seals over the open end prior to use.

In other examples, the fitting 6, 206 may be formed as a separate connector which is connected to the housing 4, 204 in the same manner as the second connector 3, 203 with a similar arrangement of movable sealing bungs which are received in a receiving chamber in the rotatable member 14, 214. In such an arrangement, the housing 4, 204 forms a central junction between the two removable (male) connectors.

To avoid unnecessary duplication of effort and repetition of text in the specification, certain features are described in relation to only one or several aspects or embodiments of the disclosure. However, it is to be understood that, where it is technically possible, features described in relation to any aspect or embodiment of the disclosure may also be used with any other aspect or embodiment of the disclosure.

The disclosure is not limited to the embodiments described herein, and may be modified or adapted without departing from the scope of the present disclosure.

1. An aseptic connector system comprising:
a first connector comprising:
a housing which defines a cavity, the housing having a first fluid passageway and a connector port which open into the cavity; and
a rotatable member rotatably mounted within the cavity and comprising a receiving chamber formed in the rotatable member and an interconnecting passageway extending through the rotatable member between a pair of openings, wherein the receiving chamber is angularly offset from the openings of the interconnecting passageway;
wherein the rotatable member is rotatable between a closed position in which the openings of the interconnecting passageway are covered and sealed by the housing and the receiving chamber is aligned with the connector port, and an open position in which the openings of the interconnecting passageway are aligned with the first fluid passageway and the connector port and the receiving chamber is angularly offset from the connector port; and
a second connector comprising:
a translatable member having a second fluid passageway which extends through the translatable member;
wherein the translatable member is translatable between a retracted position and a depressed position;
wherein the first connector comprises a first sealing bung disposed in the connector port and/or the second connector comprises a second sealing bung coupled to the translatable member;
wherein the first connector is configured to be mechanically connected to the second connector;
wherein, with the rotatable member in the closed position, the translatable member is translatable from the retracted position to the depressed position in order to force the first and/or second sealing bungs through the connector port and into the receiving chamber formed in the rotatable member; and
wherein the rotatable member can then be rotated from the closed position to the open position in order to move the first and/or second sealing bungs away from the connector port and such that the openings of the interconnecting passageway are sealed against the first and second fluid passageways so as to form a continuous fluid pathway across the first and second aseptic connectors.

2. The aseptic connector system as set forth in claim 1, wherein the first sealing bung extends from the connector port into the receiving chamber.

3. The aseptic connector system as set forth in claim 1, wherein opposing surfaces of two or more of the first sealing bung, the second sealing bung and the receiving chamber comprise complementary formations which engage when the translatable member is translated to the depressed position.

4. The aseptic connector system as set forth in claim 1, wherein an end surface of the translatable member is curved and forms a continuation of an inner surface of the housing when the translatable member is in the depressed position.

5. The aseptic connector system as set forth in claim 1, wherein an end surface of one of the first and second sealing bungs is curved and forms a continuation of an outer surface of the rotatable member when received in the receiving chamber.

6. The aseptic connector system as set forth in claim 1, wherein the first fluid passageway and the connector port are on opposite sides of the housing.

7. The aseptic connector system as set forth in claim 1, wherein the first and second fluid passageways and/or one or both of the openings of the interconnecting passageway are each provided with a seal.

8. The aseptic connector system as set forth in claim 7, wherein the or each seal is compressed in an axial direction of the seal when the rotatable member is in the closed position and expand in the axial direction when the rotatable member is in the open position to seal the interconnecting passageway against the first and second fluid passageways.

9. The aseptic connector system as set forth in claim 1, wherein the rotatable member comprises an actuation portion which extends externally to the housing so as to allow the rotatable member to be rotated.

10. The aseptic connector system as set forth in claim 1, wherein the first fluid passageway is sealed by an outer surface of the rotatable member in the closed position.

11. The aseptic connector system as set forth in claim 1, wherein the cavity is cylindrical.

12. The aseptic connector system as set forth in claim 1, wherein the second connector further comprises a connecting collar; wherein the first connector is configured to be mechanically connected to the second connector by engaging the connecting collar with the connector port; and wherein the translatable member is coupled to the connecting collar and translatable relative to the connecting collar.

13. The aseptic connector system as set forth in claim 12, wherein the second sealing bung is disposed in the connecting collar.

14. The aseptic connector system as set forth in claim 1, wherein the axis of rotation of the rotatable member passes through the interconnecting passageway.

15. An aseptic connector system as set forth in claim 1, wherein the rotatable member is rotated by 90 degrees to move between the closed position and the open position.

16. The aseptic connector system as set forth in claim 1, wherein the receiving chamber is angularly offset by 90 degrees from the openings of the interconnecting passageway.

17. The aseptic connector system as set forth in claim 1, wherein the interconnecting passageway, and first and second fluid passageways are offset from the axis of rotation.

18. The aseptic connector system as set forth in claim 17, wherein the rotatable member is rotated by 180 degrees to move between the closed position and the open position.

19. The aseptic connector system as set forth in claim 17, wherein the receiving chamber is angularly offset by 180 degrees from one of the openings of the interconnecting passageway.
